(19) United States
(12) Patent Application Publication
(10) Pub. No.: US 2007/0010781 A1
(43) Pub. Date: Jan. 11, 2007

(54) Implantable aorto-coronary sinus shunt for myocardial revascularization

(76) Inventor: Venkataramana Vijay, Tarrytown, NY (US)

Correspondence Address: Gordon & Jacobson, P.C., Suite 407, 60 Long Ridge Road, Stamford, CT, 06902, US

(21) Appl. No.: 11/168,970

(22) Filed: Jun. 27, 2005

(51) Int. Cl. A61M 5/00

(52) U.S. Cl. 604/8

(57) ABSTRACT

Embodiments of a permanent shunt are provided. According to one aspect of the invention, the shunt is a flexible biocompatible fluid directing lumen with first and second ends, with at least one of the first and second ends provided with a shape memory alloy adapted to cause the end to expand in dimension upon application of a predetermined amount of energy to cause fixation of the end of the shunt within an opening in the anatomy. According to another aspect of the invention, the shunt includes open first and second ends, a central portion therebetween, and an access port within said central portion, with the access port being smaller than the first and second ends.

### BACKGROUND OF THE INVENTION

1. Field of the Invention

This invention relates broadly to surgical methods and surgical devices. More particularly, this invention relates to methods for treating an ischemic heart by permanent perfusion of the coronary sinus and shunts therefor.

2. State of the Art

When significant diffuse stenotic coronary artery disease exists, flow through the coronary arteries is diminished. There are two conventional treatments for such a condition. One therapeutic modality to deal with this coronary arterial insufficiency is to deploy metal stents into the coronary arteries at the sites of such stenoses to improve flow. The other treatment is to surgically anastomose a detouring conduit on the coronary artery to bypass the stenosed segment; i.e., coronary artery bypass surgery. In either situation, it is not uncommon for stenoses to again appear below the treatment site, and which again require therapeutic intervention to increase blood flow through coronary arteries to nourish the myocardium.

The coronary sinus is the confluence and the final conduit for a group of cardiac veins before they empty into the right atrium. Coronary arterial blood, after passing through the myocardial bed is emptied into the right atrium in a deoxygenated state via the coronary sinus. So, in contrast to the high pressure coronary arterial system, the coronary sinus is a low pressure bed.

Previously, the coronary sinus has been used as a pathway to increase perfusion of the myocardium. An experimental modality of blocking the egress of blood flow from the coronary arterial bed, by tying off the mouth of the coronary sinus as it enters the right atrial cavity, has been employed with limited success because no new blood was delivered to the coronary sinus. Mohl W. et al., “Clinical evaluation of pressure-controlled intermittent coronary sinus occlusion: randomized trial during coronary artery surgery”, Ann Thorac Surg, 46(2):192-201 (August 1988). In another experimental animal model, anastomosis of a vein conduit (harvested from the leg) from a high pressure location on the aorta to the coronary sinus was performed. See Louis J. Acierno, The History of Cardiology, pp. 658-60 (1994) (discussing the ‘Beck I’ operation). The treatment provided long-term retrograde perfusion to the myocardium and blocked egress from the coronary sinus into the atrium. However, the procedure described is highly invasive open heart surgery, and the thinness of the coronary sinus wall is not equipped for graft anastomosis which may lead to complications such as leakage, tearing or hemorrhage.

In addition, during invasive cardiology procedures, a temporary exterior shunt from the femoral artery to the coronary sinus, accomplished on the cardiologist's catheterization table by a connecting catheter supported by a pump, has been employed to provide perfusion of the coronary artery. See Kar S et al., “Myocardial protection by diastolic coronary venous retroperfusion during PTCA” [Abstr], Proceedings of the Third International Symposium on Myocardial Protection via the Coronary Sinus (June 1988; Boston, USA) and Kar S et al., “Synchronized Coronary Venous Retroperfusion for Support and Salvage of Ischemic Myocardium During Elective and Failed Angioplasty,” J Amer. Coll. Cardio., 18(1):271-282 (1991). Similar temporary exterior shunts have also been tried by surgeons in the operating room to augment coronary bed perfusion during coronary bypass surgery. See Harinder S et al., Retrograde Coronary Sinus Perfusion for Management of Coexistent Critical Unstable Carotid and Coronary Artery Disease, Indian Heart J, 54: 717-719 (2002); Castella M et al., “Reduction of Systolic and Diastolic Dysfunction by Retrograde Coronary Sinus Perfusion During Off-Pump Surgery”, J Thoracic and Cardiovascular S, 127:1018-1025 (2004); and Harinder S, “Efficacy of Retrograde Coronary Sinus Perfusion In Off-Pump Surgery”, J Thoracic and Cardiovascular S, 129(2):476-477 (2005).

### SUMMARY OF THE INVENTION

It is therefore an object of the invention to provide methods for permanent retrograde perfusion from the aorta to the coronary sinus to provide perfusion to the heart.

It is also an object of the invention to provide methods which are less invasive than open heart surgery.

It is a further object of the invention to provide a method for permanent retrograde perfusion from the aorta to the coronary sinus which can be performed percutaneously and methods which can be performed thoracoscopically.

It is another object of the invention to provide permanently implantable shunts for implantation between the aorta and the coronary sinus.

It is an additional object of the invention to provide suitable shunts which can be implanted percutaneously and shunts which can be implanted thoracoscopically.

In accord with these objects, which will be discussed in detail below, in a minimally invasive manner, a permanent shunt is implanted to carry arterial blood from the aorta into the relatively lower pressure venous bed of the coronary sinus. Since egress from the coronary sinus into the right atrium is blocked, the higher pressure arterial blood is forced through the coronary sinus venous bed in a retrograde fashion thus accomplishing perfusion of those arterial beds that are deprived of normal antegrade coronary arterial blood flow on account of coronary arterial stenoses.

In one embodiment, the shunt is delivered percutaneously via a catheter-based delivery system. A guidewire is introduced from the lumen of the aorta, through the right atrial cavity and into the mouth of the coronary sinus, and the delivery system is delivered thereover.

In another embodiment, the shunt is delivered in a minimally invasive, preferably thoracoscopic, manner. A scope is inserted through the chest wall to gain access to the right side of the heart, the pericardium is opened, and using guidewires access to the coronary sinus and the aorta is established (from outside the right atrial cavity). The shunt is threaded into the coronary sinus and the aorta, over preferably two guidewires. Thus, most of the body of the shunt comes to lie in the pericardial sac, outside the cavity of the right atrium.

In each embodiment, the shunt must be anchored at both ends, kink-resistant, and permanently implantable. The shunt will carry high pressure arterial blood into the coronary sinus and normal egress of blood from the coronary sinus into the right atrium will be at least partially blocked resulting in retrograde perfusion of the myocardium.

Additional objects and advantages of the invention will become apparent to those skilled in the art upon reference to the detailed description taken in conjunction with the provided figures.

### BRIEF DESCRIPTION OF THE DRAWINGS

FIG. 1 is a schematic view of the heart with a percutaneous shunt delivery system according to the invention with a first embodiment of a shunt;

FIG. 2 is a schematic view of the heart with the first embodiment of the shunt implanted between the sinus of valsalva in the aorta and the mouth of the coronary sinus;

FIG. 3 is a side elevation of a second embodiment of a shunt;

FIG. 4 is a side elevation of an alternate second embodiment of the shunt;

FIG. 5 is a side elevation of yet another second embodiment of the shunt in a first configuration;

FIG. 6 is a side elevation of the shunt of FIG. 5 in a second configuration;

FIGS. 7-9 illustrate initial steps of a second method of practicing the invention;

FIG. 10 is a schematic view showing the shunt in prepared for implantation;

FIGS. 11-13 illustrate later steps of the second method of practicing the invention;

FIG. 14 is a side elevation of a third embodiment of a shunt of the invention in a first configuration; and

FIG. 15 is a side elevation of the third embodiment of the shunt of the invention in a second configuration.

### DETAILED DESCRIPTION

In accord with the invention methods are provided for introducing and implanting a permanent shunt between the aorta and the coronary sinus. The shunt then carries arterial blood from the relatively higher pressure aorta into the relatively lower pressure venous bed of the coronary sinus. As discussed in more detail below, in one embodiment, the shunt preferably has an insertion point in the lumen of the aorta and most preferably the sinus of valsalva which has fluid dynamic conditions placing it in substantially more consistent pressure than the upper tubular portion of the aorta from which prior art temporary shunts have extended. Since egress from the coronary sinus into the right atrium is blocked, the higher pressure arterial blood is forced through the coronary sinus venous bed in a retrograde fashion, thus accomplishing perfusion of those arterial beds that are deprived of normal antegrade coronary arterial blood flow on account of coronary arterial stenoses. Upon permanent retrograde perfusion, thebesian veins (micropores) within the right side of the heart become active to perfuse the myocardium of the right side of the heart. In addition, thebesian veins on the left side of the heart, which open only when the coronary sinus is blocked over an extended period of time, also open and perfuse the myocardium of the left side of the heart.

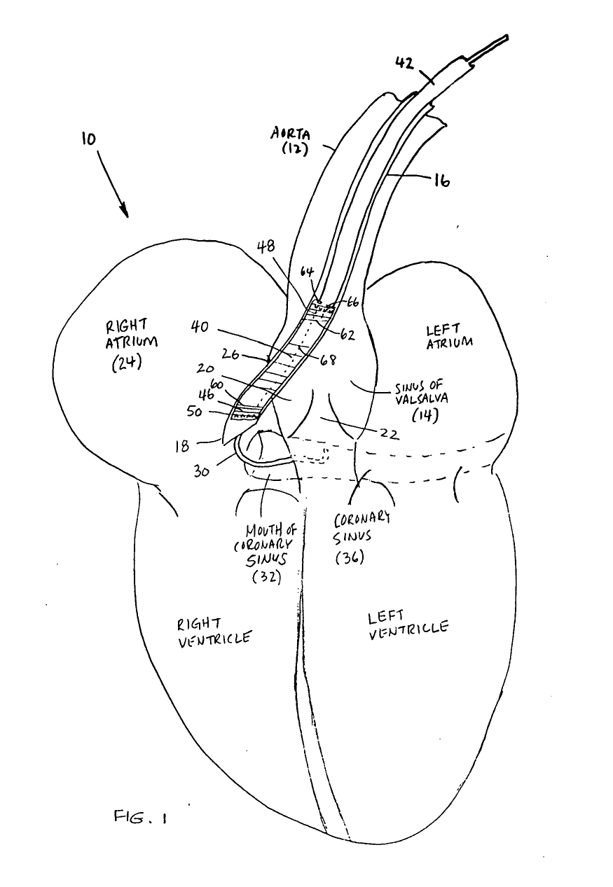

Referring now to FIG. 1, according to a first embodiment of the method in which the shunt between the aorta and the coronary sinus is implanted percutaneously in the heart 10, access to the lumen of the aorta 12 and the sinus of valsalva 14 is via the femoral, brachial, or other suitable artery. A shunt delivery system includes a guiding catheter 16 with a steerable piercing tip 18. The guiding catheter 16 is introduced to the right portion 20 or non-coronary portion 22 of the sinus of valsalva 14, as these locations will provide access to the right atrium 24. Using the tip 16 of the guiding catheter 16, the wall 26 of the sinus of valsalva 14 is pierced (in a manner similar to piercing the septum when accessing the left atrium via the right atrium) to place the catheter tip 18 within the right atrium 24. It is noted that the wall of the sinus of valsalva and the right atrial wall are nearly fused into a single structure at that level. As such, passage from the sinus of valsalva into the right atrial cavity is the straightforward matter of traversing this common wall with the sharp catheter tip.

The guiding catheter 16 has a channel that allows passage of a guidewire 30. Under ultrasound, fluoroscopy, or other radiological modality, the guidewire 30 is guided into the mouth 32 and lumen of the coronary sinus 36.

A shunt 40 is provided on a delivery catheter 42 within the guiding catheter 16. The delivery catheter 42 is threaded over the guidewire 30, into the lumen of the coronary sinus 36, via the sinus of valsalva 14 and right atrium 24. Alternately, the entire guiding catheter 16 may be introduced into the coronary sinus 36, so that an anchoring mechanism, described below, on the distal end 46 of the unexpanded end of the shunt 40 is protected from causing injury to the structures of the heart, as the shunt is threaded over the guidewire.

Once the appropriate position of the distal end 46 of the shunt 40 is confirmed in the mouth 32 of the coronary sinus 36, the anchoring mechanism at the distal end is deployed, e.g., by expanding the distal end 46 of the shunt 40, to cause the distal end of the shunt to completely occupy the inside of the mouth 32 of the coronary sinus 36. Each of the distal (coronary sinus end) and the proximal (aortic sinus of valsalva end) ends 46, 48 of the shunt are equipped with anchoring mechanisms such as barbs 50, hooks, coils, inflatable cuffs, etc. to allow proximal and distal anchoring of the shunt to the inside of their respective anatomical fixation sites.

In one embodiment, expansion is effected by self expansion, e.g., accomplished by release of tension on a resilient material as it is advanced out of the catheter sheath. Such resilient materials may be spring metals, shape memory alloys (SMA), polymers, and/or combinations thereof, covered with a polymeric or fabric material to effect a blood carrying conduit. Alternately or additionally, where the shunt has a shape memory alloy (SMA) wound or woven into a fabric or polymeric lumen, the shunt may be expandable upon application of a predetermined amount of energy. In particular, referring to FIG. 1, the shunt 40 may include a coil 60, 62 of the same shape memory alloy at each of its distal and proximal ends and separate leads 64, 66 for activation to cause respective end expansion, or different alloys activatable at different temperatures and a common activation lead or distinct activation leads for the proximal and distal ends. All activation leads preferably terminate at the proximal end for coupling to a energy source coupled to or inserted through or the delivery system. Expansion of the ends of the shunt causes fixation of the ends of the shunt within the sinus of valsalva (in distinction from expansion of the sinus of the valsalva). The central portion of the shunt may also include an SMA coil 68 which can effect expansion of the shunt to maintain lumen patency. As another alternative, the shunt may be mechanically expandable, e.g., via expansion of an internally positioned inflatable balloon which can be advanced on a balloon catheter over the guidewire 30.

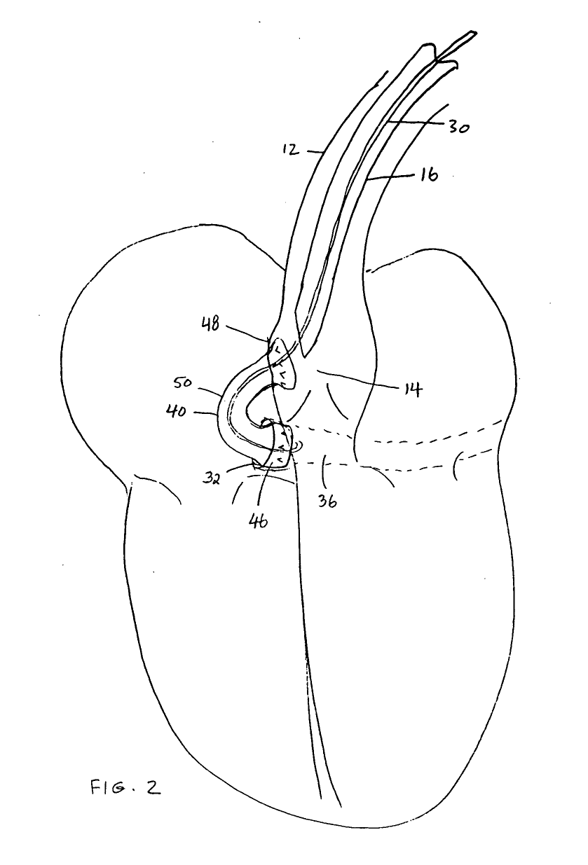

Referring to FIG. 2, once the distal end 46 of the shunt 40 is expanded to effect securement of the distal end of the shunt within the mouth 32 of the coronary sinus 36, the guiding catheter 16 is then gradually withdrawn from the coronary sinus, back into the sinus of valsalva 14 such that now the proximal end 48 of the shunt is in the sinus of valsalva. The anchoring mechanism of the proximal end of the shunt is now similarly deployed to fix it within the sinus of valsalva in a fluidtight and airtight manner.

The body 50 of the shunt (that portion between the distal and proximal ends 46, 48) is preferably designed to be patent once the delivery system is removed. Such may be via the materials used for the shunt, e.g., non-kinking skeletal frames with a polymeric or biologic covering (e.g., PTFE, woven Dacron, cell cultures, albumin, collagen, etc.), or a non-kinking polymeric tubular construct without a skeletal frame. Where a frame is used, it may be self-expanding upon the withdrawal of the catheter sheath, mechanically expandable, or expandable upon the application of a predetermined temperature (e.g., where the frame is constructed of a shape memory alloy (SMA)). Alternatively, the shunt may be an implantable natural construct (human or animal vein, artery, etc.). As yet another alternative, the central portion of the shunt may be balloon expandable and expanded to its full, open configuration after securement of the distal end or the proximal end. The guidewire 30 is removed at the end of the procedure.

The length of the shunt 40 for a percutaneous approach is preferably approximately 4 to 6 cm, the diameter of a central portion of the shunt of the preferably approximately 3 to 6 mm, the diameter at the distal end, in the expanded state, is approximately 1 to 3 cm, and the diameter at the proximal end, in the expanded state, is approximately 0.6 to 1.5 cm.

Thus, an unexpanded shunt 40 is deployed percutaneously into the coronary sinus through the aortic sinus via the right atrial cavity and anchored there distally and anchored proximally in the aorta using a catheter-based shunt delivery system. This forms a shunt, from the relatively high pressure sinus of valsalva (80-100 mmHg) with arterial blood, to the lower pressure venous coronary sinus (5-20 mmHg) via the right atrium. The shunt is an internal shunt, lying within the right atrium. Since, the mouth of the coronary sinus is completely occupied by the expanded and anchored distal opening of the conduit, egress from the coronary sinus into the right atrium is blocked.

In an alternative embodiment, after expansion of the distal end of the shunt, the distal end is secured within the mouth of the coronary sinus but egress from the coronary sinus is only partially blocked. This is advantageous and desirable in certain clinical conditions in order to avoid the temporary edema of the heart tissue that may occur with complete blockage of egress (i.e., until thebesian veins open).

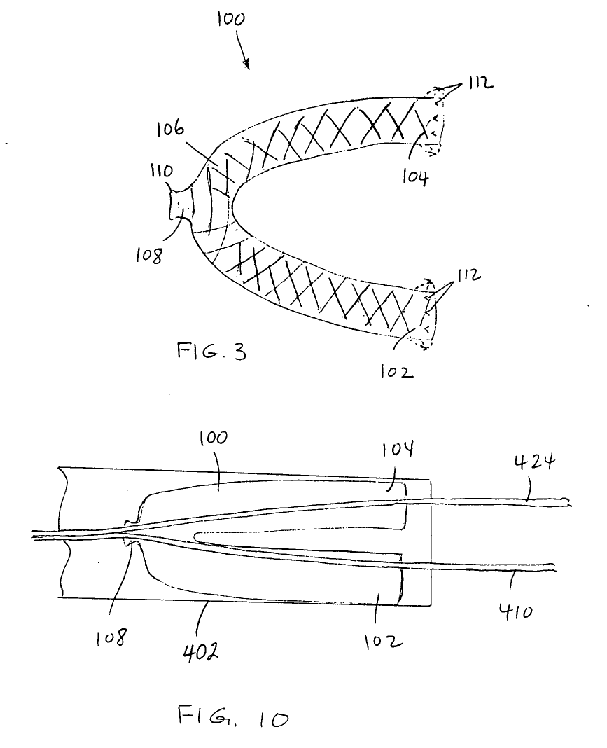

In another approach a shunt between the aorta and coronary sinus is delivered in a minimally invasive, preferably thoracoscopic manner. Prior to discussing the procedure, the shunt will now be described. Referring to FIG. 3, the shunt 100 includes first and second ends 102, 104, and a central portion 106 with a relatively small access port 108; i.e., smaller than either of the first and second ends 102, 104. The central portion 106 is preferably constructed to be patent under low pressure conditions. Nevertheless, due to the required aortic implantation location, discussed below, the shunt 100 will be subject to relatively higher pressure (on average 120 mmHg systolic pressure) than in the percutaneous approach (on average substantially constant 100 mmHg in the sinus of valsalva) and the fluid pressure of the blood within the implanted shunt should operate to maintain the patency of the shunt whether or not the shunt is provided with specialized structure specifically intended to maintain shunt patency. The access port 108 in the central portion may include a relatively resilient or rigid rim 110 which facilitates introduction of guidewires therethrough and retention of ligating clips, as discussed below. Each of the first and second ends 102, 104 includes structure 112 which can be expanded (as shown in broken lines) within the mouth of the coronary sinus and aorta, respectively, to fix the first and second ends relative thereto upon implantation, as discussed below. The shunt 100 may be any of the constructs discussed above with respect to the percutaneously deployed embodiment; i.e., structural frame in combination with a sheath, a polymeric tubular construct, or a biologic tubular construct.

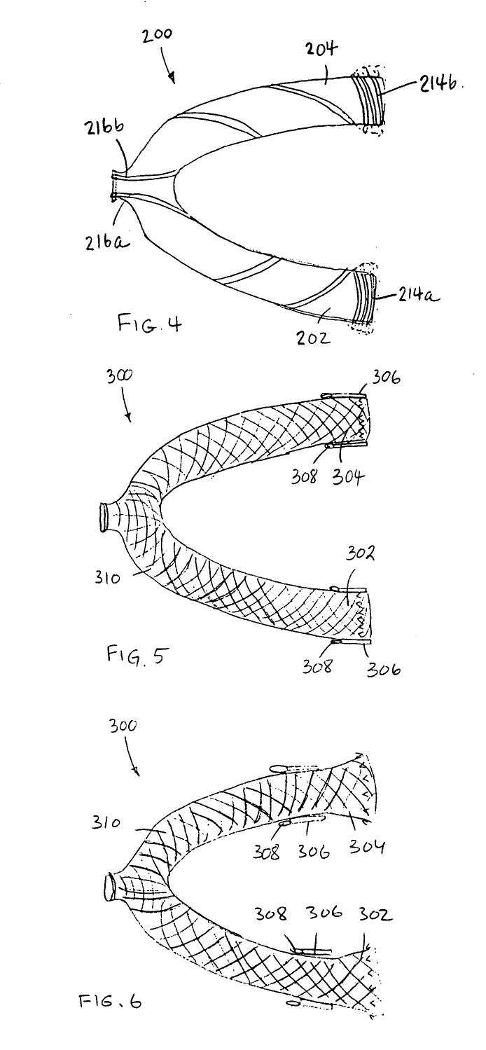

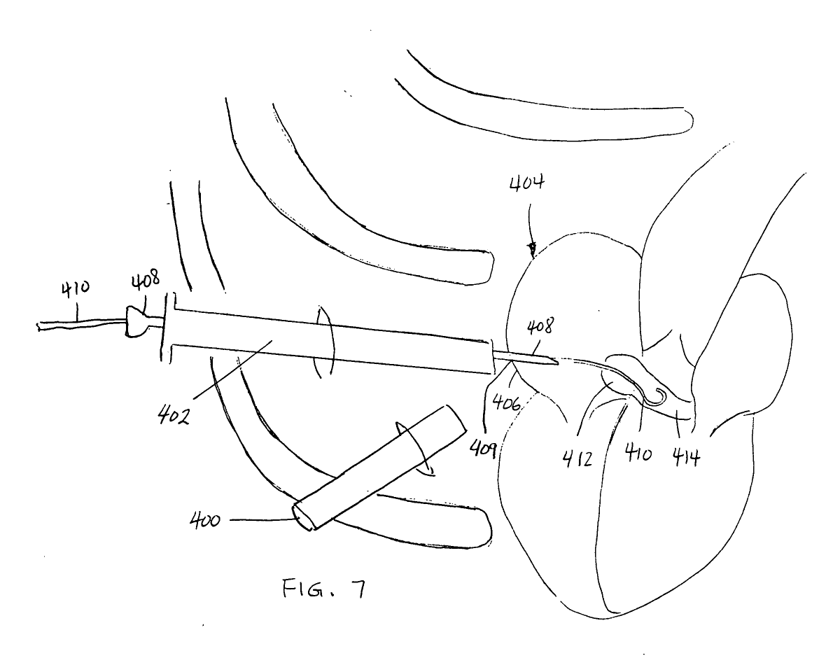

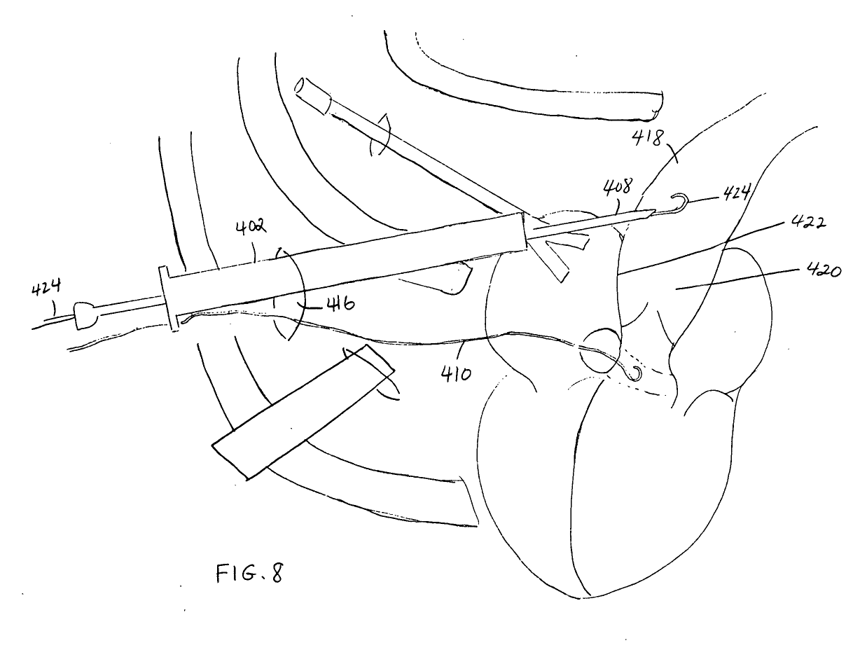

With respect to expanding the first and second ends of the shunt, the ends are preferably balloon (or otherwise mechanically) expandable. Alternatively, referring to FIG. 4, the ends 202, 204 of the shunt 200 may include SMA coils 214a, 214b or another construct with one or more leads 216a, 216b that extend to or adjacent the access port 208, facilitating activation and expansion of the ends 202, 204 (as shown in broken lines) by coupling of the leads to an energy source for application of a predetermined temperature or temperatures to cause reconfiguration (as described above in more detail with respect to shunt 40). As yet another alternatively, referring to FIGS. 5 and 6, the ends 302, 304 of the shunt 300 may be self-expandable, with retractable sleeves 306 coupled over the ends to prevent expansions of the ends during the initial stages of implantation. The sleeves 306 may be provided with loops 308 or other structure to facilitate retraction of the sleeves toward the central portion. After insertion of the ends 302, 304 into their respective implantation sites, the sleeves 306 may be retracted to cause the expansion of the ends and effect permanent retention within the implantation sites. As yet another alternative, wherein an anatomical vessel is used, the access port (a hole) will need to be created in the vessel and some means for coupling the ends within the mouth of the coronary sinus and aorta are preferably coupled to the shunt before the introducing the shunt to the implantation site.

The length of the shunt is preferably approximately 10 to 12 cm, the diameter of a central portion of the shunt of the preferably approximately 4 to 6 mm, the diameter at the distal end, in an expanded state, is approximately 1 to 3 cm, and the diameter at the proximal end, in an expanded state, is approximately 0.6 to 1.5 cm.

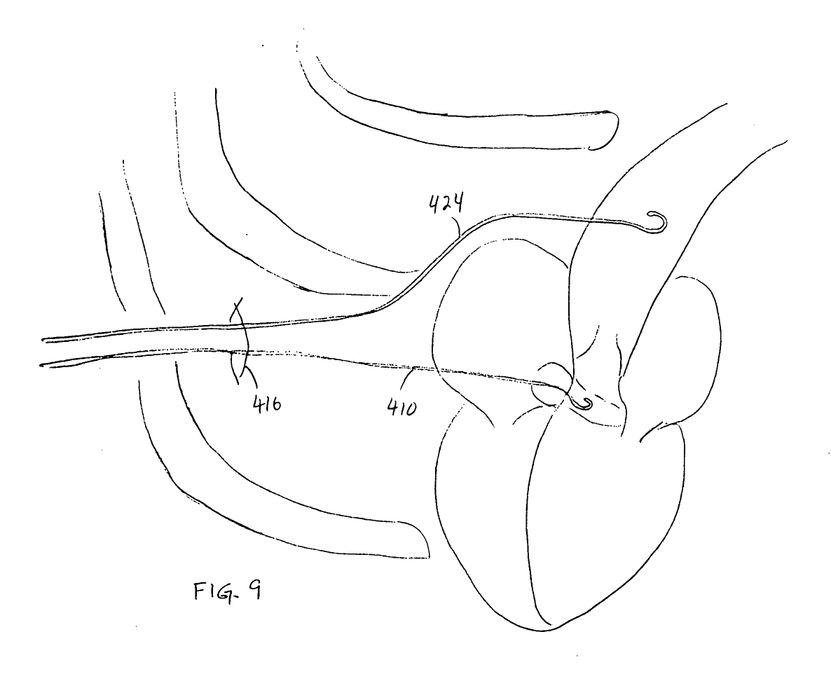

Now, in accord with another minimally invasive, preferably thoracoscopic method of the invention, the patient is anaesthetized and an appropriate amount of heparin is administered to prevent coagulation on the guidewires, the use of which is discussed below. The patient is positioned, prepped and draped. Referring to FIG. 7, preferably at the fourth, fifth or sixth intercostal space, a thoracoscope 400, a first large cannula 402 and optionally a second large cannula (not shown) are inserted through the chest wall to gain access to the right side of the heart 404. The first cannula 402 is introduced through the pericardium adjacent the right atrial wall 406. The second cannula is positioned at a distance from the thoracoscope and first cannula to define a triangular work arrangement between the scope and two cannulas and may be used if desired throughout the procedure with non-specific instruments which may facilitate the procedure, e.g., retractors, heart stabilizers, graspers, etc.

A needle 408 is introduced through the first cannula 402 and the tip of the needle is used to pierce a hole 409 through the right atrial wall 406. The location of the tip of the needle in the right atrial cavity may be confirmed by aspirating blood through the needle. Once the location is confirmed, a first guidewire 410 is introduced through the needle and directed into the mouth 412 of the coronary sinus 414. Once confirmation of the distal end of the guidewire 410 is assured, e.g., via echocardiogram, the needle 408 and first cannula 402 are withdrawn leaving the first guidewire 410 in position.

The first cannula 402 is then reinserted into the chest wall in the same hole 416 as before adjacent the first guidewire 410. The first cannula 402 is directed toward the lower ascending aorta 418. This location is chosen because access to the sinus of valsalva 420 is difficult to reach and blocked by the right atrial wall 422. Thus feeding the coronary sinus from the sinus of valsalva is less practical in a thoracoscopic approach than in a percutaneous approach. The needle 408 is inserted through the first cannula 402 to pierce the ascending aorta 418. Blood is preferably aspirated to confirm needle location within the aortic lumen. A second guidewire 424 is introduced through the needle 408 and its distal end is positioned within the aorta 418. The needle 408 and first cannula 402 are withdrawn, leaving the first and second guidewires 410, 424 exiting the chest from the same hole 416, as shown in FIG. 9.

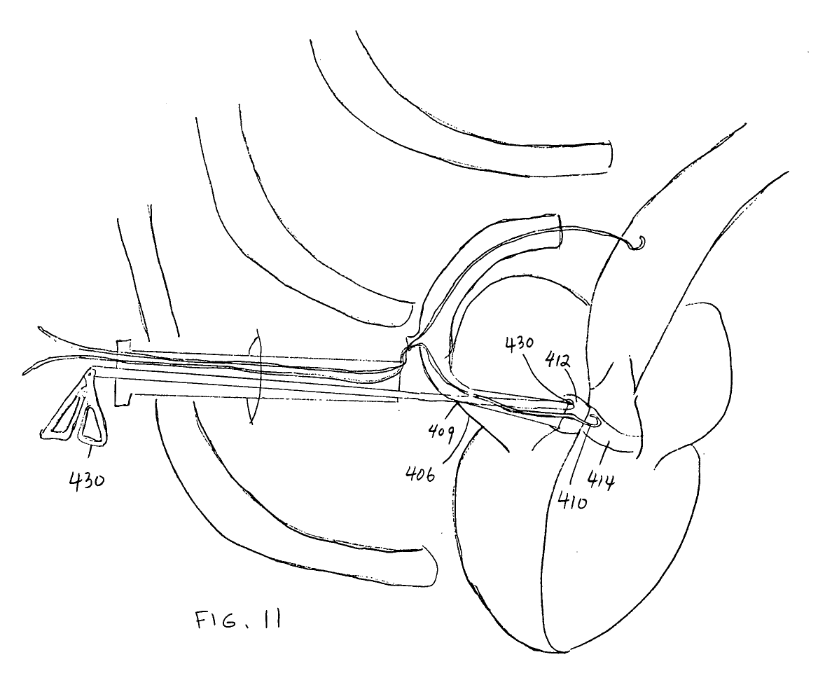

Referring to FIG. 10, the proximal ends of the first and second guidewires 410, 424 are then threaded through the first and second ends 102, 104, respectively, of the shunt 100 and out the access port 108. The shunt 100 is preferably folded into a U-shape to facilitate the insertion. The proximal ends of the guidewires ends 410, 424 and the folded shunt 100 are then positioned within the distal end of the first cannula 402, and the first cannula is reintroduced into the same hole 416 in the chest wall.

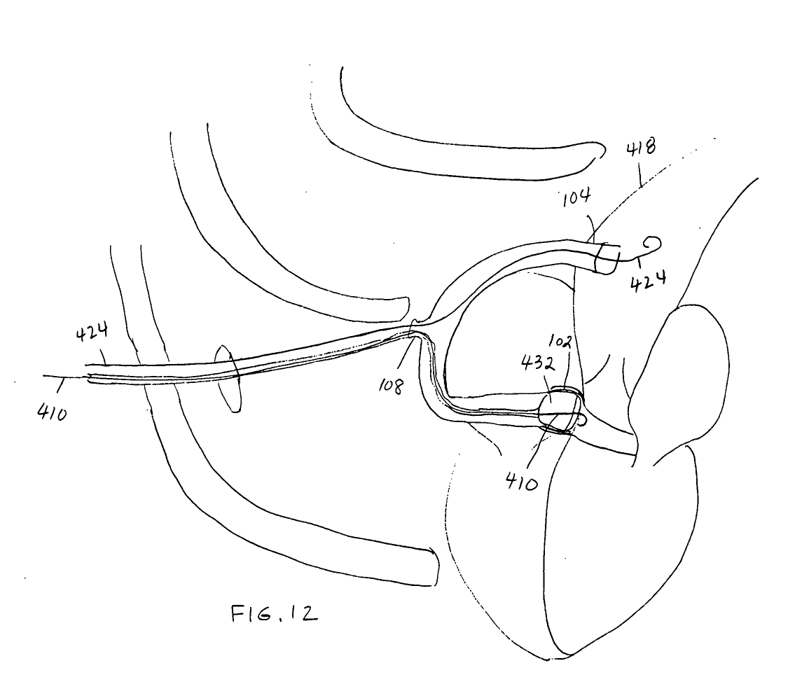

Referring to FIG. 11, a grasper/pusher instrument 430 is then introduced through the first cannula 402 and used to maneuver the first end 102 of the shunt 100 over the first guidewire 410 and through the hole 409 pierced in the right atrial wall 406 and into the mouth 412 of the coronary sinus 414. Another instrument inserted through a second cannula may facilitate this maneuver. Once the first end 102 of the shunt 100 is within the coronary sinus 414, it is expanded to effect retention therein. Where the first end is balloon expandable, a balloon catheter 432 is introduced over the first guidewire 410, through the access port 108, and into the first end 102 of the shunt 100 and inflated with fluid to cause permanent expansion of the first end 102 of the shunt (FIG. 12). The balloon catheter 432 is then removed. Where the first end is expandable by reconfiguration of an SMA element, an energy source is coupled to the lead of the first end to cause the SMA element is to reconfigure into a retaining shape/size. Where the first end is self-expandable, a grasper or other tool is used to retract the sleeve (e.g., at loops 308, FIG. 6) to allow the first end to expand. Expansion may include deployment of barbs, hooks and/or other tissue engaging structure on the outer surface of the first end of the shunt in addition to or as an alternative to expanding the diameter of the first end of the shunt. Where natural biologic tubular constructs are used, either a tissue coupling means at the first end is activated, e.g. in accord with the above, or an instrument is inserted to the implantation site which can effect the necessary anastomosis between the shunt and the mouth of the coronary sinus. Once the first end 102 of the shunt 100 is coupled within the mouth 412 of the coronary sinus 414, the first guidewire 410 is removed from the patient.

The grasper/pusher 430 is then directed to the second end 104 of the shunt 100. The second end 104 is advanced along the second guidewire 424 and inserted into the hole pierced in the aorta 418. The second end 104 is then expanded, as discussed above, to effect its retention within the aorta. The second guidewire 424 is then also removed from the patient. It is recognized that the shunt 100 is now an open conduit between the aorta 418 and the mouth 412 of the coronary sinus 414 with blood being forced under pressure in a retrograde manner into the coronary sinus. As such, there may be leakage of blood at the access port 108. Initially, this provides an avenue for any potential clot to escape. However, the access port 108 must be sealed. Therefore, referring to FIG. 13, a clip applier or other suitable ligating instrument is introduced through the first or second cannulas and operated to provide a clip 440 or other ligating element to seal the access port 108. The rim 110 on the access port 108 of the shunt 100 facilitates retention of the clip 440 or other ligating element on the shunt. Once the access port 108 is closed, the shunt 100 provides a permanent means for retrograde perfusion of the coronary sinus and the myocardium below any coronary blockage.

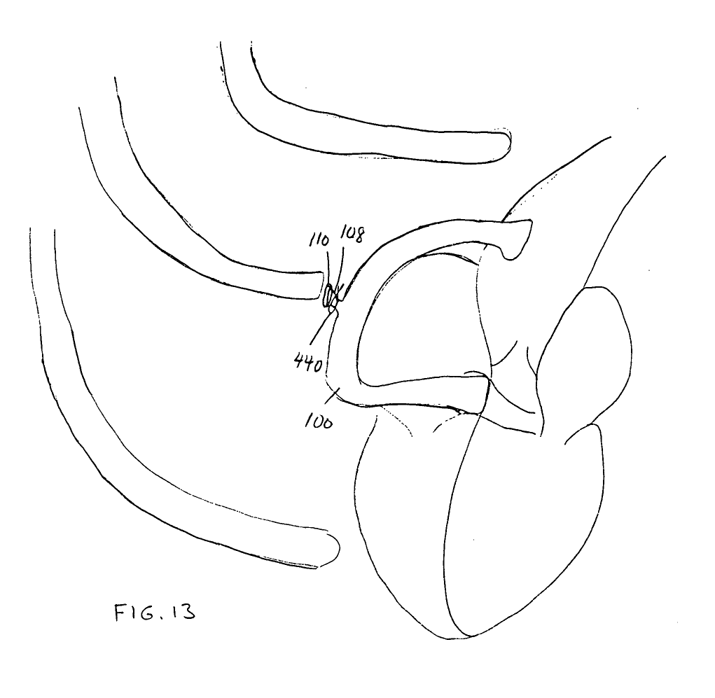

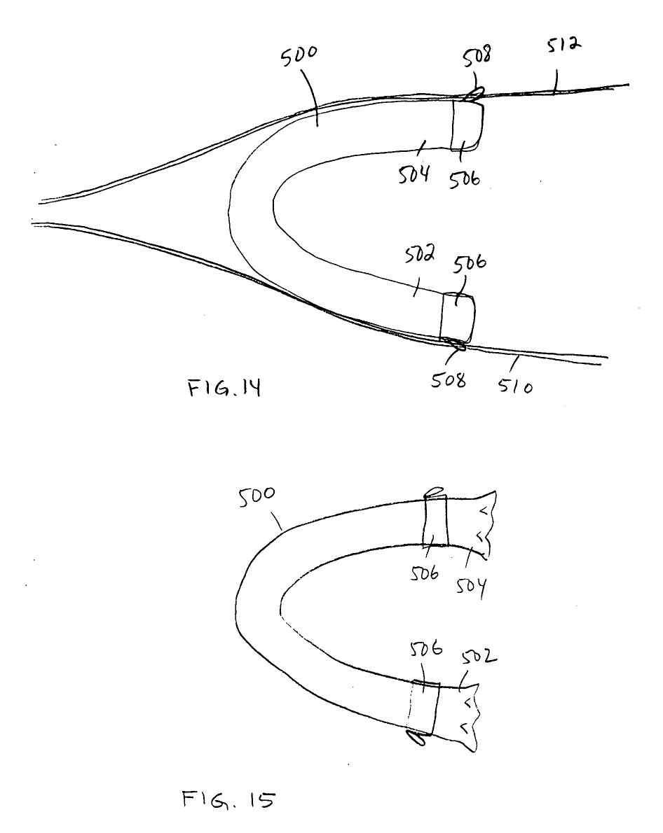

Referring to FIG. 14, a similar thoracoscopic method is provided using a shunt 500 without an access port. The first and second ends 502, 504 of the shunt 500 include retractable sleeves 506 with guidewire loops 508. The ends of the shunt may be guided on two guidewires 510, 512, as discussed above, to the appropriate location via the loops 508 (instead of through the center of the shunt). Referring to FIG. 15, once each end 502, 504 of the shunt 500 is in its intended implant location, its sleeve 506 is retracted to allow expansion of the end and retention of the end within the coronary sinus and aorta. Alternately, the shunt may be implanted thoracoscopically, and expanded with percutaneous assist.

According to an alternative minimally invasive embodiment of the invention, the first end of the shunt is modified such that, upon expansion of the first end, the first end is smaller than the mouth of the coronary sinus. As such, egress from the coronary sinus is only partially blocked. This is advantageous and desirable in certain clinical conditions in order to avoid the temporary edema of the heart tissue that may occur with complete blockage of egress (i.e., until thebesian veins open).

There have been described and illustrated herein several embodiments of shunts and methods of permanently and minimally invasively implanting shunts between the aorta and the coronary sinus. While particular embodiments of the invention have been described, it is not intended that the invention be limited thereto, as it is intended that the invention be as broad in scope as the art will allow and that the specification be read likewise. Thus, while particular shunt constructs have been disclosed, it will be appreciated that other constructs, including materials, configurations, means for end expansion and tissue retention, etc. can be used as well. It will therefore be appreciated by those skilled in the art that yet other modifications could be made to the provided invention without deviating from its spirit and scope as claimed.

1.-25. (canceled)

26. A shunt which is deployed with first and second guidewires, said shunt comprising:
open first and second ends, a central portion between the first and second ends, an access port within the central portion sized for receiving first and second guidewires, the access port being smaller than the open first and second ends.

27. A shunt according to claim 26, wherein:
said first and second ends are expandable in diameter.

28. A shunt according to claim 26, wherein:
said first and second ends are mechanically expandable from an interior of said shunt.

29. A shunt according to claim 26, wherein:
said first and second ends are provided with a shape memory alloy which is reconfigurable upon the application of a predetermined amount of thermal energy.

30. A shunt according to claim 26, wherein:
said first and second ends are self-expanding.

31. A shunt according to claim 26, wherein:
said first and second ends include tissue engaging structure.

32. A shunt according to claim 26, wherein:
said first and second ends have different maximum diameters.

33. A shunt comprising:
a flexible biocompatible fluid directing lumen with first and second ends, and a central portion therebetween, at least one of said first and second ends provided with a material adapted to cause said at least one of said first and second ends to expand in diameter upon application of a predetermined amount of thermal energy to said material.

34. A shunt according to claim 33, wherein:
said material is a shape memory alloy.

35. A shunt according to claim 33, wherein:
both of said first and second ends are provided with said material.

36. A shunt according to claim 35, wherein:
said material at said first end requires application of a first predetermined amount of energy to cause said first end to expand in diameter, and said material at said second end requires application of a second predetermined amount of energy different from said first predetermined amount of energy.

37. A shunt according to claim 36, wherein:
said first and second ends have different maximum diameters.

38. A shunt according to claim 33, wherein:
said central portion includes a shape memory alloy that is activatable to cause expansion of said central portion upon application of a predetermined amount of energy.

39. A shunt according to claim 33, wherein:
said shape memory alloy is provided in coils.

40. A shunt according to claim 39, wherein:
said shunt includes leads extending from said coils to said first end of said shunt.
